(19) United States
(12) Patent Application Publication
(10) Pub. No.: US 2025/0082570 A1
(43) Pub. Date: Mar. 13, 2025

(54) COMBINATION THERAPY FOR TREATING NON-TUBERCULOUS MYCOBACTERIAL LUNG DISEASE

(71) Applicant: Insmed Incorporated, Bridgewater, NJ (US)

(72) Inventors: Sasha ROSE, Bridgewater, NJ (US); Walter R. PERKINS, Bridgewater, NJ (US)

(21) Appl. No.: 18/372,339

(22) PCT Filed: Mar. 24, 2022
(86) PCT No.: PCT/US2022/021706

§ 371 Date: Sep. 25, 2023

Related U.S. Application Data
(60) Provisional application No. 63/165,418, filed on Mar. 24, 2021.

(51) Int. Cl. A61K 9/00; A61K 9/127; A61K 31/7028; A61K 45/06

(52) U.S. Cl. CPC A61K 9/0078; A61K 9/127; A61K 31/7028; A61K 45/06

(57) ABSTRACT

Provided herein are methods for treating NTM lung disease in a patient in need thereof. The methods comprise administering to the patient for an administration period (i) a liposomal amikacin composition and (ii) a second active agent that is synergistic with amikacin against the NTM. The liposomal amikacin composition comprises amikacin, or a pharmaceutically acceptable salt thereof, encapsulated in a plurality of liposomes and lipid component of the liposomes consists of one or more electrically neutral lipids. Administration of the liposomal amikacin composition comprises aerosolizing the composition to provide an aerosolized composition comprising a mixture of free amikacin and liposomal complexed amikacin, and administering the aerosolized composition via a nebulizer to the lungs of the patient. The second active agent is administered to the patient orally, parenterally or via inhalation, and can be administered at the same or different dosing intervals as the liposomal amikacin composition.

### CROSS REFERENCE TO RELATED APPLICATION

This application is a National Stage of International Patent Application Number PCT/US2022/021706 filed Mar. 24, 2022, which claims priority from U.S. Provisional Application No. 63/165,418, filed Mar. 24, 2021, the disclosure of which is incorporated by reference herein in its entirety.

### BACKGROUND OF THE INVENTION

Non-tuberculous mycobacterium (NTM) pulmonary infection in the susceptible host can lead to potentially severe morbidity and eventually mortality among those affected. As infection rates are rising, NTM lung disease represents an emerging public health concern in the United States. NTM are ubiquitous in the environment. Over 80% of NTM lung disease in the US are due to Mycobacterium avium complex (MAC). In addition, M. kansasii, M abscessus, and M. fortuitum are regularly isolated in subjects diagnosed with NTM lung disease.

NTM lung disease caused by Mycobacterium avium complex (MAC) is a potentially life-threatening and progressively destructive disease that is associated with symptoms of productive cough, fatigue, shortness of breath, fever, weight loss, lung function decline, and hemoptysis. It often complicates other chronic debilitating underlying lung diseases such as bronchiectasis or COPD. When NTM lung disease occurs in patients without underlying lung comorbidities, it has been implicated in progressive lung disease.

The prevalence of NTM lung disease in the United States has more than doubled in the last 15 years. The American Thoracic Society (ATS)/Infectious Disease Society of America (IDSA) reported 2-year period prevalence of pulmonary NTM infections is 8.6/100,000 persons. The prevalence of pulmonary NTM infections increases with age with 20.4/100,000 in those at least 50 years of age and is especially prevalent in females (median age: 66 years; female: 59%).

Arikayce® (amikacin liposome inhalation suspension or ALIS), is the first FDA approved treatment for NTM lung disease. Specifically, ALIS is an aminoglycoside antibacterial indicated in adults who have limited or no alternative treatment options, for the treatment of Mycobacterium avium complex (MAC) lung disease as part of a combination antibacterial drug regimen in patients who do not achieve negative sputum cultures after a minimum of 6 consecutive months of a multidrug background regimen therapy.

Although an approved therapy for NTM lung disease exists, and other antibiotics have been used historically to treat NTM lung disease, available therapies may be poorly tolerated, and may have significant adverse events. The present invention addresses this and other needs by providing methods for treating NTM lung disease in patients in need thereof.

### SUMMARY OF THE INVENTION

Provided herein are methods for treating NTM lung disease in a patient in need thereof. In one aspect, the method comprises administering to the patient for an administration period, (i) a liposomal amikacin composition and (ii) a second active agent that is synergistic with amikacin against the NTM. The liposomal amikacin composition comprises amikacin, or a pharmaceutically acceptable salt thereof, encapsulated in a plurality of liposomes and lipid component of the liposomes consists of one or more electrically neutral lipids. Administration of the liposomal amikacin composition comprises aerosolizing the composition to provide an aerosolized composition comprising a mixture of free amikacin and liposomal complexed amikacin, and administering the aerosolized composition via a nebulizer to the lungs of the patient. The second active agent is administered to the patient orally, parenterally or via inhalation, and can be administered at the same or different dosing intervals as the liposomal amikacin composition.

In one embodiment, synergy is assessed by the Fractional Inhibitory Concentration Index (FICI) value.

In one embodiment, the second active agent is a carbapenem. In a further embodiment, the second active agent is imipenem, doripenem, biapenem or tebipenem.

In yet another embodiment, the second active agent is rifabutin, rifampin, RV40, clofazimine, bedaquiline, or cefdinir.

In another embodiment, the second active agent is imipenem, rifabutin, rifampin, RV40, clofazimine, bedaquiline, cefdinir, doripenem, biapenem, or tebipenem.

The liposomal amikacin composition and second active agent are administered to the patient in need of treatment during an administration period. The administration period is measured from the time that a patient received both the liposomal amikacin composition and the second active agent (T₁) to the time point where both the liposomal amikacin composition and the second active agent are no longer administered (T₂). During the administration period, the liposomal amikacin composition and the second active agent need not be administered for the same amount of time, via the same route, or via the same dosing schedule.

The second active agent, i.e., an active agent that is synergistic with amikacin against the NTM, can be administered to the patient orally, parenterally or locally via inhalation. For example, in one embodiment, the second active agent is administered orally. In another embodiment, the second active agent is administered parenterally. In a further embodiment, the second active agent is administered intravenously. In even another embodiment, second active agent is administered via inhalation, e.g., via a nebulizer, metered dose inhaler (MDI) or dry powder inhaler (DPI).

The NTM lung disease, in one embodiment, is caused by a M. avium, M. abscessus, or M. avium complex (MAC) (M. avium and M. intracellulare) pulmonary infection. In embodiments where the NTM lung disease is caused by a M. avium pulmonary infection, the M. avium can be M. avium subsp. hominissuis (MAH). In one embodiment, the pulmonary NTM lung disease is caused by M. avium complex (MAC) (M. avium and M. intracellulare). In one embodiment, the pulmonary NTM infection is a pulmonary recalcitrant NTM infection.

In one embodiment, the NTM lung disease treated by the methods provided herein is caused by Mycobacterium abscessus or Mycobacterium avium complex. In one or more of the preceding embodiments, the patient is a cystic fibrosis (CF) patient, a bronchiectasis patient, an asthma patient or a COPD patient.

In one embodiment, the liposomal amikacin composition is a dispersion (i.e., a suspension) of liposomes. The liposomal portion of the composition comprises a lipid component that includes one or more electrically neutral lipids. In a further embodiment, the electrically neutral lipids comprise a phosphatidylcholine and a sterol (e.g., dipalmitoylphosphatidylcholine (DPPC) and cholesterol). In one embodiment, the amikacin in the liposomal amikacin composition is amikacin sulfate.

In one embodiment, the method for treating the NTM disease comprises in part, aerosolizing the liposomal amikacin composition to provide an aerosolized composition, and administering the aerosolized composition to the lungs of the patient in need of treatment; wherein the aerosolized pharmaceutical composition comprises a mixture of free amikacin and liposomal complexed amikacin. In a further embodiment, the lipid component of the liposomal amikacin composition comprises a phosphatidylcholine and a sterol (e.g., DPPC and cholesterol). In a further embodiment, the amikacin is present as a pharmaceutically acceptable salt. In even a further embodiment, the amikacin is amikacin sulfate.

The methods provided herein, in one embodiment, comprise achieving a negative NTM culture subsequent to the administration of the liposomal amikacin composition and the second active agent. In one embodiment, the methods provided herein comprise achieving an NTM culture conversion to negative subsequent to the administration period. The patient subject to the methods provided herein, in one embodiment, achieves a negative NTM sputum culture faster than a patient administered either the liposomal amikacin composition or the second active agent alone. In one embodiment, the patient subject to the methods provided herein culture converts, i.e., achieves three (3) consecutive negative NTM cultures prior to a patient administered either the liposomal amikacin composition or the second active agent alone, wherein the sputum cultures are obtained from both patients at the same time points.

In yet another embodiment of the invention provided herein, during the administration period or subsequent to the administration period, the patient shows improvement in one or more respiratory symptoms, as measured by a QOL-B respiratory domain score, as compared to the one or more respiratory symptoms of the patient prior to the administration period.

### BRIEF DESCRIPTION OF THE FIGURES

FIG. 1 is a schematic of one method for carrying out a checkerboard minimum inhibitory concentration (MIC) assay.

### DETAILED DESCRIPTION OF THE INVENTION

Nontuberculous mycobacteria are organisms found in the soil and water that can cause serious lung disease in susceptible individuals, for which there are currently limited effective treatments and no approved therapies. The prevalence of NTM disease is reported to be increasing, and according to reports from the American Thoracic Society is believed to be greater than that of tuberculosis in the U.S. According to the National Center for Biotechnology Information, epidemiological studies show that presence of NTM infection is increasing in developing countries, perhaps because of the implementation of tap water. Women with characteristic phenotype are believed to be at higher risk of acquiring NTM infection along with patients with defects on cystic fibrosis transmembrane conductance regulators. Generally, high risk groups with NTM lung disease for increased morbidity and mortality are those with cavitary lesions, low BMI, advanced age, and a high comorbidity index.

NTM lung disease is often a chronic condition that can lead to progressive inflammation and lung damage, and is characterized by bronchiectasis and cavitary disease. NTM infections often require lengthy hospital stays for medical management. Treatment usually involves multi-drug regimens that can be poorly tolerated and have limited effectiveness, especially in patients with severe disease or in those who have failed prior treatment attempts. According to a company-sponsored patient chart study conducted by Clarity Pharma Research, approximately 50,000 patients suffering from NTM lung disease visited physician offices in the U.S. during 2011.

Management of NTM lung disease caused by nontuberculous mycobacterial infection includes lengthy multidrug regimens, which are often associated with drug toxicity and suboptimal outcomes. Achieving NTM culture negativity is one of the objectives of treatment and represents the most clinically important microbiologic endpoint in patients with NTM lung infection.

The present invention described herein is directed in part to methods for treating NTM lung disease with a liposomal amikacin composition and a second active agent which is synergistic with amikacin. Without wishing to be bound by theory, it is thought that the synergistic combination of antiinfectives provides greater efficacy in certain NTM lung disease treatment methods, as described herein, compared to the use of a liposomal amikacin composition. In addition, the combination therapy approach, without wishing to be bound by theory, is thought to delay antimicrobial resistance, thereby providing a more effective treatment option than the use of one of the antiinfective agents alone, or a combination of antiinfectives that are not synergistic.

In one aspect, the present invention provides methods for treating a nontuberculous mycobacterial (NTM) lung disease in a patient in need thereof. The method, in one embodiment comprises administering to the patient during an administration period (i) a liposomal amikacin composition comprising amikacin, or a pharmaceutically acceptable salt thereof, encapsulated in a plurality of liposomes, wherein the lipid component of the plurality of liposomes consists of one or more electrically neutral lipids and (ii) a second active agent that is synergistic with amikacin.

In one embodiment, the one or more neutral lipids in the liposomal amikacin composition comprise a phospholipid and a sterol. In a further embodiment, the phospholipid is a phosphatidylcholine. In even a further embodiment, the phosphatidylcholine is dipalmitoylphosphatidylcholine (DPPC). In even a further embodiment, the sterol is cholesterol. In one embodiment, the second active agent that is synergistic with amikacin is imipenem, rifabutin, rifampin, RV40, clofazimine, levofloxacin, moxifloxacin, bedaquiline, cefdinir, doripenem, biapenem, tebipenem, ethambutol or tetrandrine. In another embodiment, the second active agent that is synergistic with amikacin is imipenem, rifabutin, rifampin, RV40, clofazimine, bedaquiline, cefdinir, doripenem, biapenem, or tebipenem. In another embodiment, the second active agent that is synergistic with amikacin is rifabutin, rifampin, RV40, clofazimine, bedaquiline, or cefdinir.

The NTM lung disease treated by the methods provided herein, in one embodiment, is caused by M. avium (e.g., M. avium subsp. hominissuis (MAH)), M. abscessus, M. chelonae, or M. avium complex (MAC) (M. avium and M. intracellulare). In one embodiment, the NTM lung disease is caused by M. avium complex (MAC) (M. avium and M. intracellulare). In one embodiment, the NTM lung infection is a recalcitrant nontuberculous mycobacterial lung infection.

In one embodiment, the NTM lung disease is caused by a M abscessus, M kansasii, M. fortuitum, Mycobacterium avium or a M. avium complex (MAC) lung infection. In one embodiment, the patient in need of treatment is administered the liposomal amikacin composition via inhalation, and the second active agent orally, intravenously or via inhalation. In one embodiment, the liposomal amikacin composition is administered once per day in a single dosing session. In even a further embodiment, the NTM lung disease is caused by a M. avium complex.

The NTM lung disease, in one embodiment, is associated with cavitary lesions. In one embodiment, the NTM lung disease is a nodular NTM lung disease. In one embodiment, the NTM lung disease is nodular with minimal cavitary lesions.

In one embodiment, the NTM lung disease is caused by Mycobacterium abscessus or Mycobacterium avium complex (MAC) lung infection. In one embodiment, the NTM lung disease is caused by a recalcitrant nontuberculous mycobacterial lung infection.

In embodiments of the NTM lung disease treatment methods described herein, the liposomal amikacin composition and the second active agent are administered to a patient in need thereof during the administration period. The administration period is measured from the time that a patient receives both the liposomal amikacin composition and the second active agent (T₁) to the time point where both the liposomal amikacin composition and the second active agent are no longer administered (T₂). During the administration period, the liposomal amikacin composition and second active agent need not be administered at the same time, at the same dosing intervals, or for the same duration.

During the administration period, the liposomal amikacin composition and the second active agent need not be administered for the same amount of time, via the same route, or via the same dosing schedule. For example, treatment with the liposomal amikacin composition and the second active agent can begin at the same time point (T₁), and the second active agent administration can be discontinued prior to the discontinuation of liposomal amikacin administration. In this scenario, the administration period is measured from T₁ until the liposomal amikacin composition administration is discontinued (T₂). In another embodiment, administration of the liposomal amikacin composition and the second active agent begins at the same time point (T₁) and ends at the same time point (T₂).

The administration period, in one embodiment, is at least 1 month, at least 2 months, at least 3 months, at least 4 months, at least 5 months, at least 6 months, at least 9 months, at least 12 months, at least 15 months, at least 18 months or at least 24 months. The administration period, in another embodiment, is from about 6 months to about 24 months, or from about 6 months to about 18 months or from about 6 months to about 12 months.

In one embodiment, the administration period is from about 30 days to about 400 days, e.g., from about 45 days to about 300 days, or from about 45 days to about 270 days, or from about 80 days to about 200 days. In another embodiment, the administration period is from about 80 days to about 400 days, or from about 90 days to about 400 days, or from about 100 days to about 400 days. In another embodiment, the administration period is from about 100 days to about 500 days.

The term “treating” includes: (1) preventing or delaying the appearance of clinical symptoms of the state, disorder or condition developing in the subject that may be afflicted with or predisposed to the state, disorder or condition but does not yet experience or display clinical or subclinical symptoms of the state, disorder or condition; (2) inhibiting the state, disorder or condition (i.e., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a subject to be treated is either statistically significant or at least perceptible to the subject or to the physician.

Treatment includes a therapeutic response that a user (e.g., a clinician) will recognize as an effective response to the combination therapy. The therapeutic response in one embodiment, is a reduction, inhibition, delay or prevention in growth of or reproduction of one or more NTM, or the killing of one or more NTM. In one embodiment, during the administration period or subsequent to the administration period, the patient achieves an NTM negative culture. The NTM culture is prepared, in one embodiment, from a sputum sample obtained from the patient. In another embodiment, the patient achieves NTM culture conversion to negative, during the administration period or subsequent to the administration period. Culture conversion to negative as provided herein, refers to three consecutive negative NTM cultures. The three consecutive cultures can be prepared from patient samples that are obtained at spaced apart intervals, for example, two-week or monthly intervals.

“Effective amount” means an amount of liposomal amikacin composition and a second synergistic active agent used in the present invention sufficient to result in the desired therapeutic response.

The term “about,” as used herein, refers to plus or minus ten percent of the object that “about” modifies.

As described throughout, the methods and compositions described herein are used to treat NTM lung disease and include a combination of a liposomal amikacin composition and a second active agent. The second active agent, i.e., an active agent that is synergistic with amikacin against the NTM, can be administered to the patient orally, parenterally or locally via inhalation. As such, the second active agent can be administered via the same route (inhalation) or a different route, as compared to the liposomal amikacin composition. If administered via inhalation, in one embodiment, the second active agent can be in the same composition as the liposomal amikacin composition, or a different composition. In one embodiment, the second active agent is administered orally. In another embodiment, the second active agent is administered parenterally. In a further embodiment, the second active agent is administered intravenously. In even another embodiment, second active agent is administered via inhalation, e.g., via a nebulizer or dry powder inhaler (DPI).

In one embodiment, the second active agent is a carbapenem. In a further embodiment, the second active agent is imipenem, doripenem, biapenem or tebipenem.

In yet another embodiment, the second active agent is rifabutin, rifampin, RV40, clofazimine, levofloxacin, moxifloxacin, bedaquiline, cefdinir, ethambutol or tetrandrine. In a further embodiment, the second active agent is rifabutin, rifampin, RV40, clofazimine, bedaquiline, or cefdinir.

In another embodiment, the second active agent is imipenem, rifabutin, rifampin, RV40, clofazimine, levofloxacin, moxifloxacin, bedaquiline, cefdinir, doripenem, biapenem, tebipenem, ethambutol or tetrandrine. In a further embodiment, the second active agent is imipenem, rifabutin, rifampin, RV40, clofazimine, bedaquiline, cefdinir, doripenem, biapenem, or tebipenem.

Amikacin, in one embodiment, is present in the liposomal amikacin composition as amikacin base, or as a pharmaceutically acceptable salt of amikacin, for example, amikacin sulfate or amikacin disulfate.

A “pharmaceutically acceptable salt” includes both acid and base addition salts. In one embodiment, a pharmaceutically acceptable salt is a pharmaceutically acceptable acid addition salt which retains the biological effectiveness and properties of the free base, and which is not biologically or otherwise undesirable. A pharmaceutically acceptable acid addition salt may be formed with an inorganic acid, such as, but not limited to, hydrochloric acid (HCl), hydrobromic acid, sulfuric acid, nitric acid, phosphoric acid, or may be formed with an organic acid, such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecyl sulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid (e.g., as lactate), lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, acetic acid (e.g., as acetate), tartaric acid, thiocyanic acid, p-toluenesulfonic acid, trifluoroacetic acid (TFA), and undecylenic acid. In one embodiment, the pharmaceutically acceptable salt is a HCl, TFA, lactate, or acetate salt. In another embodiment, the pharmaceutically acceptable salt is a sulfate salt.

The liposomal amikacin composition, as provided herein, includes (i) amikacin or a pharmaceutically acceptable salt thereof, encapsulated in a plurality of liposomes, (ii) amikacin, or a pharmaceutically acceptable salt thereof complexed to the lipid bilayers or surface of the plurality of liposomes, or (iii) a combination thereof. A “liposomal complexed amikacin” includes embodiments where (i) the amikacin is encapsulated in liposomes, (ii) the amikacin is associated with the liposomal bilayer via a covalent or non-covalent bond, (iii) the amikacin is present in the aqueous phase or the hydrophobic bilayer phase or at the interfacial headgroup region of the liposomal bilayer of the liposomes or (iv) a combination of any of the foregoing. In the embodiments provided herein, substantially all the amikacin in the liposomal amikacin composition is complexed with the liposomes. For example, ≥95%, ≥96%, ≥97%, or ≥98% of the amikacin is complexed with the liposomes prior to the administration of the composition.

The methods provided herein comprise in part, administering via inhalation, to a patient in need thereof, a composition comprising amikacin, or pharmaceutically acceptable salt thereof encapsulated in a plurality of liposomes. In one embodiment, the lipid component of the plurality of liposomes comprises one or more electrically neutral lipids. In even a further embodiment, the electrically neutral lipids comprise a sterol and a phospholipid. In even a further embodiment the sterol is cholesterol and the phospholipid is a net neutral phosphatidylcholine. In a further embodiment, the phosphatidylcholine is dipalmitoyl phosphatidylcholine (DPPC).

The lipid component of the plurality of liposomes can include one or more synthetic, semi-synthetic or a naturally occurring lipids, including a phospholipid, tocopherol, sterol, fatty acid, or a combination thereof. In one embodiment, the lipid component of the plurality of liposomes consists of electrically neutral lipids. In a further embodiment, the lipid component comprises DPPC and cholesterol.

In one embodiment, at least one phospholipid is present in the lipid component of the plurality of liposomes. The phospholipid, in one embodiment, is electrically net neutral. In one embodiment, the phospholipid is a phosphatidylcholine (PC), phosphatidylglycerol (PG), phosphatidylinositol (PI), phosphatidylserine (PS), phosphatidylethanolamine (PE), phosphatidic acid (PA); a soya counterpart of one of the foregoing or the hydrogenated egg and soya counterpart of one of the foregoing (e.g., hydrogenated egg PC, hydrogenated egg PC).

In one embodiment, the lipid component of the plurality of liposomes includes dipalmitoylphosphatidylcholine (DPPC), a major constituent of naturally-occurring lung surfactant. In one embodiment, the lipid component of the plurality of liposomes comprises DPPC and cholesterol, or consists essentially of DPPC and cholesterol, or consists of DPPC and cholesterol. In a further embodiment, the DPPC and cholesterol have a mole ratio in the range of from about 19:1 (DPPC:cholesterol) to about 1:1 (DPPC:cholesterol), or about 9:1 (DPPC:cholesterol) to about 1:1 (DPPC:cholesterol), or about 4:1 (DPPC:cholesterol) to about 1:1 (DPPC:cholesterol), or about 2:1 (DPPC:cholesterol) to about 1:1 (DPPC:cholesterol). In even a further embodiment, the DPPC and cholesterol have a mole ratio of about 2:1 (DPPC:cholesterol), about 1.5:1 (DPPC:cholesterol) or about 1:1 (DPPC:cholesterol). In yet a further embodiment, the DPPC and cholesterol have a mole ratio of about 2:1 (DPPC:cholesterol).

In even a further embodiment, the DPPC and cholesterol have a weight ratio of about 2:1 (DPPC:cholesterol), about 1.5:1 (DPPC:cholesterol) or about 1:1 (DPPC:cholesterol). In yet a further embodiment, the DPPC and cholesterol have a weight ratio of about 2:1 (DPPC:cholesterol).

Other examples of lipids for use with the methods and compositions described herein include, but are not limited to, dimyristoylphosphatidycholine (DMPC), dimyristoylphosphatidylglycerol (DMPG), dipalmitoylphosphatidcholine (DPPC), dipalmitoylphosphatidylglycerol (DPPG), distearoylphosphatidylcholine (DSPC), distearoylphosphatidylglycerol (DSPG), dioleoylphosphatidyl-ethanolamine (DOPE), mixed phospholipids such as palmitoylstearoylphosphatidyl-choline (PSPC), and single acylated phospholipids, for example, mono-oleoyl-phosphatidylethanolamine (MOPE).

In one embodiment, the lipid component of the plurality of liposomes comprises a sterol. In a further embodiment, the at least one lipid component comprises a sterol and a phospholipid, or consists essentially of a sterol and a phospholipid, or consists of a sterol and a phospholipid (e.g., a neutral phosphatidylcholine such as DPPC). Sterols for use with the invention include, but are not limited to, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate, lanosterol sulfate and tocopherols. The tocopherols can include tocopherols, esters of tocopherols including tocopherol hemi-succinates, salts of tocopherols including tocopherol hydrogen sulfates and tocopherol sulfates. The term “sterol compound” includes sterols, tocopherols and the like.

In one embodiment, at least one cationic lipid is provided in the lipid component of the plurality of liposomes of the liposomal complexed amikacin. Cationic lipids amendable for use with the present invention include but are not limited to ammonium salts of fatty acids, phospholipids, and glycerides. The fatty acids include fatty acids of carbon chain lengths of 12 to 26 carbon atoms that are either saturated or unsaturated. Some specific examples include, but are not limited to, myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio) propane (DOTAP), and combinations thereof.

In one embodiment, at least one anionic lipid (negatively charged lipid) is provided in the lipid component of the plurality of liposomes, present in the liposomal amikacin compositions described herein, for use in the method of treating an NTM pulmonary infection in a patient in need thereof. The negatively-charged lipids which can be used include phosphatidyl-glycerols (PGs), phosphatidic acids (PAs), phosphatidylinositols (PIs) and the phosphatidyl serines (PSs). Examples include but are not limited to DMPG, DPPG, DSPG, DMPA, DPPA, DSPA, DMPI, DPPI, DSPI, DMPS, DPPS, DSPS and combinations thereof.

Without wishing to be bound by theory, phosphatidylcholines, such as DPPC, aid in the uptake of the amikacin by the cells in the lung (e.g., the alveolar macrophages) and helps to maintain the amikacin in the lung. The negatively charged lipids such as the PGs, PAs, PSs and PIs, in addition to reducing particle aggregation, are thought to play a role in the sustained activity characteristics of the inhalation composition as well as in the transport of the composition across the lung (transcytosis) for systemic uptake. The sterol compounds, without wishing to be bound by theory, are thought to affect the release characteristics of the composition.

Liposomes are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer) or a combination thereof. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

The lipid component to amikacin ratio by weight (weight ratios are also referred to herein as “lipid:amikacin” or “lipid-to-amikacin weight ration”) in the liposomal amikacin composition, in one embodiment, is 3:1 or less, 2.5:1.0 or less, 2:1 or less, 1.5:1 or less, 1:1 or less or 0.75:1 or less. In one embodiment, the lipid-to-amikacin weight ratio in the liposomal amikacin composition provided herein is 0.7:1.0 or about 0.7:1.0 by weight. In another embodiment, the lipid-to-amikacin weight ratio weight ratio in the liposomal amikacin composition provided herein is 0.75:1 (lipid:amikacin) or less (by weight). In one embodiment, the lipid-to-amikacin weight ratio is from about 0.10:1.0 to about 1.25:1.0, from about 0.25:1.0 to about 1.25:1.0, from about 0.50:1.0 to about 1.25:1.0 or from about 0.6:1 to about 1.25:1.0. In another embodiment, the lipid-to-amikacin weight ratio is from about 0.60:1.0 (lipid:amikacin) to about 0.79:1.0 (lipid:amikacin).

The lipid-to-amikacin weight ratio in the liposomal amikacin composition provided herein in another embodiment, is less than 3:1 (lipid:amikacin), less than 2.5:1.0 (lipid:amikacin), less than 2.0:1.0 (lipid:amikacin), less than 1.5:1.0 (lipid:amikacin), or less than 1.0:1.0 (lipid:amikacin). In yet another embodiment, the lipid to amikacin weight ratio is from about 0.6:1.0 (lipid:amikacin) to about 0.8:1.0 (lipid:amikacin).

In one embodiment, the liposomal amikacin composition is amikacin liposome inhalation suspension (ALIS), marketed under the trade name Arikayce®.

In one embodiment, the lipid-to-amikacin weight ratio in the liposomal amikacin composition provided herein is 0.7:1.0 (lipid:amikacin), about 0.7:1.0 (lipid:amikacin), from about 0.5:1.0 (lipid:amikacin) to about 0.8:1.0 (lipid:amikacin) or from about 0.6:1.0 (lipid:amikacin) to about 0.8:1.0 (lipid:amikacin). In a further embodiment, the liposomes provided herein are small enough to effectively penetrate a bacterial biofilm. In one embodiment, the mean diameter of the plurality of liposomes, as measured by light scattering is from about 150 nm to about 350 nm, or from about 200 nm to about 400 nm, or from about 250 nm to about 400 nm, or from about 250 nm to about 300 nm, or from about 200 nm to about 300 nm. In even a further embodiment, the mean diameter of the plurality of liposomes, as measured by light scattering is from about 260 nm to about 280 nm.

In one embodiment, the liposomal compositions described herein are manufactured by one of the methods set forth in U.S. Patent Application Publication No. 2013/0330400 or U.S. Pat. No. 7,718,189, each of which is incorporated by reference in its entirety for all purposes. In another embodiment, the liposomal amikacin composition is manufactured by one of the methods set forth in WO/2019/213398, incorporated by reference herein in its entirety. In yet another embodiment, the liposomal amikacin composition is manufactured by one of the methods set forth in WO/2019/191627, incorporated by reference herein in its entirety. Other liposomal manufacturing methods are known in the art and can be employed herein to manufacture a liposomal amikacin composition. In one embodiment, one or more of the methods described in U.S. Patent Application Publication No. 2008/0089927, incorporated by reference herein in its entirety, are used herein to produce the liposomal amikacin composition.

In one embodiment, the liposomes are formed by dissolving one or more lipids in an organic solvent forming a lipid solution, and the amikacin coacervate forms from mixing an aqueous solution of the amikacin with the lipid solution. In a further embodiment, the organic solvent is ethanol. In even a further embodiment, the lipid solution comprises a phospholipid and a sterol, e.g., DPPC and cholesterol.

In one embodiment, liposomes are produced by sonication, extrusion, homogenization, swelling, electroformation, inverted emulsion or a reverse evaporation method. Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Papahadjopoulos et al., U.S. Pat. No. 4,235,871, discloses preparation of oligolamellar liposomes by reverse phase evaporation. Each of the methods is amenable for use with the present invention.

Unilamellar vesicles can be produced from MLVs by a number of techniques, for example, the extrusion techniques of U.S. Pat. Nos. 5,008,050 and 5,059,421. Sonication and homogenization can be used to produce smaller unilamellar liposomes from larger liposomes (see, for example, Chapman et al., “Physical studies of phospholipids. X. The effect of sonication of aqueous dispersions of egg yolk lecithin,” Biochim Biophys Acta. 163(2):255-61 (1968), incorporated herein by reference in its entirety for all purposes).

The liposome preparation of Bangham et al. (J. Mol. Biol. 13, 1965, pp. 238-252) involves suspending phospholipids in an organic solvent which is then evaporated to dryness leaving a phospholipid film on the reaction vessel. Next, an appropriate amount of aqueous phase is added, the mixture is allowed to “swell,” and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys. Acta. 135, 1967, pp. 624-638), and large unilamellar vesicles.

Techniques for producing large unilamellar vesicles (LUVs), such as, reverse phase evaporation, infusion procedures, and detergent dilution, can be used to produce liposomes for use in the pharmaceutical compositions provided herein. A review of these and other methods for producing liposomes may be found in the text Liposomes, Marc Ostro, ed., Marcel Dekker, Inc., New York, 1983, Chapter 1, which is incorporated herein by reference. See also Szoka, Jr. et al., (Ann. Rev. Biophys. Bioeng. 9, 1980, p. 467), which is also incorporated herein by reference in its entirety for all purposes.

Other techniques for making liposomes include those that form reverse-phase evaporation vesicles (REV), U.S. Pat. No. 4,235,871. Another class of liposomes that may be used is characterized as having substantially equal lamellar solute distribution. This class of liposomes is denominated as stable plurilamellar vesicles (SPLV) as defined in U.S. Pat. No. 4,522,803, and includes monophasic vesicles as described in U.S. Pat. No. 4,588,578, and frozen and thawed multilamellar vesicles (FATMLV) as described above.

A variety of sterols and their water soluble derivatives such as cholesterol hemisuccinate have been used to form liposomes; see, e.g., U.S. Pat. No. 4,721,612. Mayhew et al., PCT Publication No. WO 1985/00968, described a method for reducing the toxicity of drugs by encapsulating them in liposomes comprising alpha-tocopherol and certain derivatives thereof. Also, a variety of tocopherols and their water soluble derivatives have been used to form liposomes, and can be used to manufacture the liposomal amikacin described herein. For example, the methods disclosed in PCT Publication No. 1987/02219, incorporated by reference herein in its entirety, can be employed herein.

The liposomal amikacin composition, in one embodiment, pre-nebulization, comprises liposomes with a mean diameter, that is measured by a light scattering method, of approximately 150 nm to approximately 400 nm, for example, in the range about 150 nm to about 350 nm. In one embodiment, the mean diameter of the liposomes in the composition is about 150 nm to about 300 nm, about 200 nm to about 300 nm, about 210 nm to about 290 nm, about 220 nm to about 280 nm, about 230 nm to about 280 nm, about 240 nm to about 280 nm, about 250 nm to about 280 nm or about 260 nm to about 280 nm.

In the methods provided herein, a patient in need of NTM lung disease treatment is co-administered during an administration period, (i) a liposomal amikacin composition via inhalation, for example, via a nebulizer and (ii) a second active agent which is synergistic with amikacin. In one embodiment, the patient is administered about 450 mg, about 500 mg, about 550 mg, about 560 mg, about 570 mg, about 580 mg, about 590 mg, about 600 mg or about 610 mg amikacin once daily during the administration period. In another embodiment, the amount of amikacin provided in the composition and administered once daily to the patient in need of treatment, is from about 500 mg to about 600 mg, or from about 500 mg to about 650 mg, or from about 525 mg to about 625 mg, or from about 550 mg to about 600 mg. In one embodiment, the amount of amikacin administered to the subject is about 560 mg and is provided in an 8 mL composition. In one embodiment, the amount of amikacin administered to the subject is about 590 mg and is provided in an 8 mL-10 mL composition, for example, an 8 mL-9 mL liposomal suspension in 1.5% NaCl. In one embodiment, the amount of amikacin in the liposomal amikacin composition administered once daily to the patient during the administration period is about 590 mg and is provided in an 8 mL-9 mL composition. the amikacin provided in the composition is about 450 mg, about 500 mg, about 550 mg, about 560 mg, about 570 mg, about 580 mg, about 590 mg, about 600 mg or about 610 mg. In another embodiment, the amount of amikacin provided in the composition is from about 500 mg to about 650 mg, or from about 525 mg to about 625 mg, or from about 550 mg to about 600 mg. In one embodiment, the amount of amikacin administered to the patient once daily during the administration period is about 590 mg, and is provided in an 8 mL-9 mL composition for nebulization. In one embodiment, the amount of amikacin administered to the patient once daily during the administration period is about 590 mg, and is provided in an 8.2 mL-8.6 mL composition for nebulization. In one embodiment, the liposomal amikacin composition is an 8.3 mL-8.5 mL composition.

In one embodiment, the liposomal amikacin composition provided herein comprises about 60 mg/mL to about 80 mg/mL amikacin, for example, from about 65 mg/mL to about 80 mg/mL amikacin, from about 65 mg/mL to about 75 mg/mL amikacin. In one embodiment, the liposomal amikacin composition provided herein comprises about 60 mg/mL amikacin, about 65 mg/mL amikacin, about 70 mg/mL amikacin, about 75 mg/mL amikacin, about 80 mg/mL amikacin, about 85 mg/mL amikacin, or about 90 mg/mL amikacin. In a further embodiment, the amikacin is amikacin sulfate.

The liposomal amikacin composition is administered as an aerosol via nebulization to the patient in need of treatment. In one embodiment, the liposomal amikacin composition is administered once per day in a single dosing session during the administration period. In another embodiment, the method comprises administering the liposomal amikacin composition to a patient in need thereof every other day or every three days during the administration period. In yet another embodiment, the method comprises administering the liposomal amikacin composition to a patient in need thereof twice per day during the administration period.

During the administration period, in one embodiment, the patient in need of NTM lung disease treatment is administered the liposomal amikacin composition via nebulization, and about 500 mg to about 1000 mg amikacin is administered daily in a single dosing session, for example, about 500 mg amikacin to about 700 mg amikacin (e.g., about 590 mg amikacin) is administered daily, in a single dosing session, during the administration period.

In one embodiment, the liposomal amikacin composition is provided in an about 8 mL suspension. In one embodiment, the density of the liposomal amikacin composition is about 1.05 gram/mL; and in one embodiment, approximately 8.4 grams of the liposomal amikacin composition per dose is present in the composition of the invention. In a further embodiment, the entire volume of the composition is administered to a subject in need thereof.

In one embodiment, the liposomal amikacin composition provided herein comprises amikacin and a lipid component comprising at least one phospholipid and cholesterol. In a further embodiment, the liposomal amikacin composition comprises amikacin sulfate, DPPC and cholesterol. In one embodiment, the liposomal amikacin composition is a composition provided in Table 1 or Table 2, below.

TABLE 1
Liposomal Amikacin Compositions

| Composition A (pH 6.0-7.0) | | Composition D (pH ~6.5 | |
|---|---|---|---|
| Component | Concentration | Component | Concentration |
| Amikacin | 60-80 mg/mL | Amikacin | 62-79 mg/mL |
| Phospholipid | 25-40 mg/mL | Phospholipid | 23-38 mg/mL |
| Sterol | 10-20 mg/mL | Sterol | 12-20 mg/mL |
| Salt | 0.5%-5.0% | Salt | ~1.5% |
| Composition B (pH 6.0-7.0) | | Composition E (pH ~6.5) | |
| Component | Concentration | Component | Concentration |
| Amikacin | 62-78 mg/mL | Amikacin | 65-75 mg/mL |
| Sulfate | | Sulfate | |
| DPPC | 26-38 mg/mL | DPPC | 30-40 mg/mL |
| Cholesterol | 12-20 mg/mL | Cholesterol | 12-20 mg/mL |
| NaCl | 0.5%-2.0% | NaCl | ~1.5% |
| Composition C (pH 6.0-7.0) | | Composition F (pH ~6.5) | |
| Component | Concentration | Component | Concentration |
| Amikacin | 62-79 mg/mL | Amikacin | 62-79 mg/mL |
| Sulfate | | Sulfate | |
| DPPC | 26-40 mg/mL | DPPC | 26-40 mg/mL |
| Cholesterol | 12-19 mg/mL | Cholesterol | 12-19 mg/mL |
| NaCl | 0.5%-3.0% | NaCl | ~1%-2% |

TABLE 2
Exemplary Liposomal Amikacin
Composition

| Amikacin Sulfate | ~70 mg/mL |
|---|---|
| DPPC | ~30-35 mg/mL |
| Cholesterol | ~15-17 mg/mL |
| NaCl | ~1.5% |

In embodiments where the liposomal amikacin and/or the second active agent is delivered via inhalation, the inhalation can be conducted via a nebulizer. The nebulizer provides an aerosol mist of the composition for delivery to the lungs of the patient.

An “aerosol,” as used herein, is a gaseous suspension of liquid particles. The aerosol provided herein comprises particles of the liposomal dispersion.

A “nebulizer” or an “aerosol generator” is a device that converts a liquid into an aerosol of a size that can be inhaled into the respiratory tract. Pneumonic, ultrasonic, electronic nebulizers, e.g., passive electronic mesh nebulizers, active electronic mesh nebulizers and vibrating mesh nebulizers are amenable for use with the invention if the particular nebulizer emits an aerosol with the required properties, and at the required output rate.

The process of pneumatically converting a bulk liquid into small droplets is called atomization. The operation of a pneumatic nebulizer requires a pressurized gas supply as the driving force for liquid atomization. Ultrasonic nebulizers use electricity introduced by a piezoelectric element in the liquid reservoir to convert a liquid into respirable droplets. Various types of nebulizers are described in Respiratory Care, Vol. 45, No. 6, pp. 609-622 (2000), the disclosure of which is incorporated herein by reference in its entirety. The terms “nebulizer” and “aerosol generator” are used interchangeably throughout the specification. “Inhalation device,” “inhalation system” and “atomizer” are also used in the literature interchangeably with the terms “nebulizer” and “aerosol generator.”

“Mass median diameter” or “MMD” is determined by laser diffraction or impactor measurements, and is the average particle diameter by mass.

“Mass median aerodynamic diameter” or “MMAD” is normalized regarding the aerodynamic separation of aqua aerosol droplets and is determined impactor measurements, e.g., the Anderson Cascade Impactor (ACI) or the Next Generation Impactor (NGI). The gas flow rate, in one embodiment, is 28 Liter per minute by the Anderson Cascade Impactor (ACI) and 15 Liter per minute by the Next Generation Impactor (NGI). “Geometric standard deviation” or “GSD” is a measure of the spread of an aerodynamic particle size distribution.

In one embodiment, the liposomal amikacin composition and/or the second active agent is delivered to a patient in need of treatment via a nebulizer selected from the group consisting of an electronic mesh nebulizer, pneumonic (jet) nebulizer, ultrasonic nebulizer, breath-enhanced nebulizer and a breath-actuated nebulizer. In a further embodiment, the nebulizer is portable.

In one embodiment, the liposomal amikacin composition is delivered to a patient in need of treatment via a nebulizer, once a day in single dosing sessions. In even a further embodiment, the nebulizer is one of the nebulizers described in U.S. Patent Application Publication No. 2013/0330400, incorporated by reference herein in its entirety for all purposes.

The principle of operation of a pneumonic nebulizer is generally known to those of ordinary skill in the art and is described, e.g., in Respiratory Care, Vol. 45, No. 6, pp. 609-622 (2000), incorporated by reference herein in its entirety. Briefly, a pressurized gas supply is used as the driving force for liquid atomization in a pneumatic nebulizer. Compressed gas is delivered, which causes a region of negative pressure. The solution to be aerosolized is then delivered into the gas stream and is sheared into a liquid film. This film is unstable and breaks into droplets because of surface tension forces. Smaller particles, i.e., particles with the MMAD and FPF properties described above, can then be formed by placing a baffle in the aerosol stream. In one pneumonic nebulizer embodiment, gas and solution is mixed prior to leaving the exit port (nozzle) and interacting with the baffle. In another embodiment, mixing does not take place until the liquid and gas leave the exit port (nozzle). In one embodiment, the gas is air, O₂ and/or CO₂.

In one embodiment, droplet size and output rate can be tailored in a pneumonic nebulizer which can be used in the methods provided herein. However, consideration should be paid to the composition being nebulized, and whether the properties of the composition (e.g., % associated amikacin) are altered due to the modification of the nebulizer. For example, in one embodiment, the gas velocity and/or pharmaceutical composition velocity is modified to achieve the output rate and droplet sizes of the present invention. Additionally, or alternatively, the flow rate of the gas and/or solution can be tailored to achieve the droplet size and output rate of the invention. For example, an increase in gas velocity, in one embodiment, decreased droplet size. In one embodiment, the ratio of pharmaceutical composition flow to gas flow is tailored to achieve the droplet size and output rate of the invention. In one embodiment, an increase in the ratio of liquid to gas flow increases particle size.

In one embodiment, a pneumonic nebulizer output rate is increased by increasing the fill volume in the liquid reservoir. Without wishing to be bound by theory, the increase in output rate may be due to a reduction of dead volume in the nebulizer. Nebulization time, in one embodiment, is reduced by increasing the flow to power the nebulizer. See, e.g., Clay et al. (1983). Lancet 2, pp. 592-594 and Hess et al. (1996). Chest 110, pp. 498-505.

In one embodiment, a reservoir bag is used to capture aerosol during the nebulization process, and the aerosol is subsequently provided to the subject via inhalation. In another embodiment, the nebulizer provided herein includes a valved open-vent design. In this embodiment, when the patient inhales through the nebulizer, nebulizer output is increased. During the expiratory phase, a one-way valve diverts patient flow away from the nebulizer chamber.

In one embodiment, the nebulizer provided herein is a continuous nebulizer. In other words, refilling the nebulizer with the pharmaceutical composition while administering a dose is not needed. Rather, the nebulizer has at least an 8 mL capacity, at least a 8.4 mL capacity, at least an 8.6 mL capacity, at least an 8.8 mL capacity, at least a 9 mL capacity, at least a 9.4 mL capacity, at least an 9.6 mL capacity, at least an 9.8 mL capacity, or at least a 10 mL capacity.

In one embodiment, the nebulizer provided herein does not use an air compressor and therefore does not generate an air flow. In one embodiment, aerosol is produced by the aerosol head which enters the mixing chamber of the device. When the patient inhales, air enters the mixing chamber via one-way inhalation valves in the back of the mixing chamber and carries the aerosol through the mouthpiece to the patient. On exhalation, the patient's breath flows through the one-way exhalation valve on the mouthpiece of the device. In one embodiment, the nebulizer continues to generate aerosol into the mixing chamber which is then drawn in by the subject on the next breath—and this cycle continues until the nebulizer medication reservoir is empty.

In one embodiment, the nebulization time of the liposomal amikacin composition provided herein, e.g., ALIS, during a dosing session, is less than 20 minutes, less than 18 minutes, less than 16 minutes or less than 15 minutes. In one embodiment, the nebulization time of the liposomal amikacin composition is less than 15 minutes or less than 13 minutes. In one embodiment, the nebulization time of an effective amount of a liposomal amikacin composition provided herein during a dosing session is about 13 minutes. In another embodiment, the nebulization time of a liposomal amikacin composition provided herein during a dosing session is from about 13 minutes to about 17 minutes, or from about 13 minutes to about 16 minutes, or from about 13 minutes to about 15 minutes.

In one embodiment, the liposomal amikacin composition described herein is administered once daily to a patient in need thereof.

In one embodiment, the liposomal amikacin composition comprises from about 550 mg to about 600 mg amikacin, DPPC and cholesterol, and the lipid-to-amikacin weight ratio of the composition is 0.75:1.0 (lipid:amikacin) or less, e.g., about 0.7:1.0 (lipid:amikacin) or about 0.5:1.0 (lipid:amikacin) to about 0.8:1.0 (lipid component:amikacin).

In one embodiment, prior to nebulization of the liposomal amikacin composition, about 95% to about 100% of the amikacin present in the composition is liposomal complexed. In a further embodiment, the amikacin is an amikacin sulfate. In another embodiment, prior to nebulization, about 95% to about 99% or about 96% to about 99% of the amikacin present in the composition is liposomal complexed. In a further embodiment, the amikacin is amikacin sulfate. In another embodiment, ≥97% of the amikacin present in the liposomal amikacin composition is liposomal complexed prior to nebulization. In a further embodiment, the amikacin is amikacin sulfate.

Upon nebulization of the liposomal amikacin composition described herein, i.e., for administration to a patient in need of treatment of an NTM lung disease, an aerosolized composition is formed, and in one embodiment, the mass median aerodynamic diameter (MMAD) of the aerosolized composition is about 1.0 μm to about 4.2 μm as measured by the Anderson Cascade Impactor (ACI). In one embodiment, the MMAD of the aerosolized composition is about 3.2 μm to about 4.2 μm as measured by the AC. In one embodiment, the MMAD of the aerosolized composition is about 1.0 μm to about 4.9 μm as measured by the Next Generation Impactor (NGI). In a further embodiment, the MMAD of the aerosolized composition is about 4.4 μm to about 4.9 μm as measured by the NGI.

The fine particle fraction (FPF) of the aerosolized composition, in one embodiment, is greater than or equal to about 64%, as measured by the Anderson Cascade Impactor (ACI), or greater than or equal to about 51%, as measured by the Next Generation Impactor (NGI). In one embodiment, the FPF of the aerosolized composition is greater than or equal to about 70%, as measured by the ACI, greater than or equal to about 510%, as measured by the NGI, or greater than or equal to about 60%, as measured by the NGI.

Upon nebulization, as provided above, the liposomes in the liposomal amikacin composition leak amikacin from the liposomes. In one embodiment, upon nebulization, from about 20% to about 45% of the liposomal complexed amikacin is released from the liposomes, thereby providing an aerosol comprising a mixture of free amikacin and liposomal complexed amikacin. In a further embodiment, the amikacin is amikacin sulfate. In a further embodiment, from about 25% to about 45%, or from about 30% to about 40% of the liposomal complexed amikacin is released from the liposomes, thereby providing an aerosol comprising a mixture of free amikacin and liposomal complexed amikacin. In a further embodiment, the amikacin is amikacin sulfate.

In another embodiment, the amount of liposomal complexed amikacin post-nebulization is from about 55% to about 80%, e.g., from about 55% to about 75%, or from about 55% to about 70% or from about 60% to about 70%. These percentages are also referred to herein as “percent associated amikacin post-nebulization.” In one embodiment, the percent associated amikacin post-nebulization is from about 55% to about 75%, or example, from about 60% to about 70%. In a further embodiment, the amikacin is amikacin sulfate.

In one embodiment, the percent associated amikacin post-nebulization is measured by reclaiming the aerosol from the air by condensation in a cold-trap, and subsequently assaying for free and associated amikacin.

As provided throughout, the methods described herein comprise in part, administering to a patient in need of NTM lung disease treatment during an administration period, an effective amount of (i) a liposomal amikacin composition via inhalation, and (ii) a second active agent that is synergistic with amikacin. The second active agent can be delivered together with the liposomal amikacin, i.e., in the same composition, or in a separate composition. When present in a different composition, the second active agent, can be delivered to the patient in need of treatment orally, parenterally or locally via inhalation during the administration period.

Synergy can be assessed according to the ordinary skill in the art, for example, via in vitro MIC assays and calculating a Fractional Inhibitory Concentration Index (FICI) value. FICI is determined by the following equation. Table 3 provides the means for interpreting FICI values for amikacin and the second active agent.

$$\frac{A}{MICA} + \frac{B}{MICB} = FICA + FICB = F I C I$$

where A and B are the MIC of Antiinfective-X (amikacin) and Antiinfective-Y (second active agent) in combination (in a single well), and MICA and MICB are the MIC of amikacin (MICA) and the second active agent (MICB) individually.

TABLE 3

| FIC value | Interpretation |
|---|---|
| <0.5 | Synergy |
| >4 | Antagonism |
| 0.5-4 | Additive or indifference |

In one embodiment, the second active agent is a carbapenem. In a further embodiment, the second active agent is imipenem, doripenem, biapenem or tebipenem.

In yet another embodiment, the second active agent is rifabutin, rifampin, RV40, clofazimine, levofloxacin, moxifloxacin, bedaquiline, cefdinir, ethambutol or tetrandrine. In a further embodiment, the second active agent is rifabutin, rifampin, RV40, clofazimine, bedaquiline, or cefdinir.

In another embodiment, the second active agent is imipenem, rifabutin, rifampin, RV40, clofazimine, levofloxacin, moxifloxacin, bedaquiline, cefdinir, doripenem, biapenem, tebipenem, ethambutol or tetrandrine. In a further embodiment, the second active agent is imipenem, rifabutin, rifampin, RV40, clofazimine, bedaquiline, cefdinir, doripenem, biapenem, or tebipenem.

Each of the aforementioned active agents has been shown to be synergistic with amikacin against NTM, as described in further detail in the Example section herein.

The second active agent compound is administered in one embodiment, via inhalation. In a further embodiment, the second active agent is present in the liposomal amikacin composition. For example, the second active agent is provided as free drug in the composition. Alternatively, the second active agent is administered via inhalation in a separate composition. The second active agent, in one embodiment, is administered via inhalation as a “free” antiinfective. In other words, in this embodiment, the second active agent is not liposomally complexed. However, in another embodiment, the second active agent is liposomally complexed and administered via inhalation.

In one embodiment, the second active agent is administered orally to the patient in need of NTM lung disease treatment.

In one embodiment, the second active agent is administered parenterally to the patient in need of NTM lung disease treatment. In a further embodiment, the second active agent is administered intravenously to the patient in need of NTM lung disease treatment.

Rifabutin, in one embodiment, is the second active agent used in one of the methods described herein. In one embodiment, rifabutin is administered orally or intravenously to the patient in need of treatment. In one embodiment, rifabutin is administered orally once-daily during the administration period to the patient. In another embodiment, rifabutin is administered orally twice-daily during the administration period to the patient. Rifabutin, in one embodiment, is administered at a dose of 150 mg or 300 mg daily during the administration period.

Rifampin, a semisynthetic antibiotic derivative of rifamycin SV, in one embodiment, is the second active agent used in one of the methods described herein. In one embodiment, rifabutin is administered orally or intravenously to the patient in need of treatment. The dose of rifampin, in one embodiment, is from about 300 mg to about 600 mg per administration. In a further embodiment, the dose of rifampin is 300 mg or 600 mg per administration. In a further embodiment, rifabutin is administered once-daily during the administration period to the patient.

The vancomycin derivative, RV40, in one embodiment, is used in one of the methods described herein as the second active agent. Methods for making RV40 are disclosed in PCT publication No. WO 2018/217800, incorporated by reference herein in its entirety. RV40, in one embodiment is administered intravenously to the patient in need of treatment. In another embodiment, RV40 is administered via inhalation, e.g., via a DPI or nebulizer.

In another embodiment, the second active agent is clofazimine. In one embodiment, clofazimine is administered orally. In another embodiment, clofazimine is administered via inhalation, via a nebulizer or DPI. Various inhalation formulations of clofazimine have been described and are amenable for use with the present methods. For example, the formulations described in PCT Publication WO 2019/110099, incorporated by reference herein in its entirety, can be used in the methods provided herein. In another embodiment, a clofazimine dry powder formulation for administration via DPI can be used. See, e.g., Brunaugh et al. (2017). Mol. Pharmaceutics 14, pp. 4019-4031, incorporated by reference herein in its entirety. Administration can be carried out once-daily or twice-daily during the administration period.

The fluoroquinolone levofloxacin, in one embodiment, is used as the second active agent in one of the NTM lung disease treatment methods provided herein. Levofloxacin, in one embodiment, is administered orally or intravenously to the patient in need of treatment. For example, levofloxacin can be administered orally or intravenously as described in the prescribing information for Levaquin® (levofloxacin).

The fluoroquinolone moxifloxacin (marketed under the trade name Avelox®), in one embodiment, is used as the second active agent in one of the NTM lung disease treatment methods provided herein. Moxifloxacin, in one embodiment, is administered orally or intravenously to the patient in need of treatment. Administration in one embodiment, is carried out once daily during the administration period. In a further embodiment, the patient is administered moxifloxacin orally, intravenously, or sequentially (intravenous followed by oral). In a further embodiment, the patient is administered 400 mg once daily during the administration period.

Bedaquiline, in one embodiment, is the second active agent used in one of the NTM lung disease treatment methods provided herein. Bedaquiline in one embodiment, is administered orally to the patient in need of treatment. In a further embodiment, the patient is administered from about 100 mg to about 400 mg once daily during the administration period. In another embodiment, the patient is administered 400 mg once daily for 2 weeks, followed by 200 mg 3 times per week for 22 weeks or more.

In another embodiment, bedaquiline is administered via inhalation. Various inhalation formulations of bedaquiline have been described and are amenable for use with the present methods. For example, the formulations described in PCT Publication Nos. WO 2020/123336 and WO 2019/193609, each incorporated by reference herein in their entirety for all purposes, can be used in the methods provided herein. In another embodiment, a bedaquiline dry powder formulation for administration via DPI can be used. See, e.g., Momin et al. (2019). Pharmaceutics 11, 502, doi:10.3390/pharmaceutics11100502, incorporated by reference herein in its entirety.

The cephalosporin antibiotic cefdinir, can also be used as the second active agent in one of the methods described herein. Cefdinir is marketed under the trade name Omnicef®. In one embodiment, cefdinir is administered to the patient once or twice daily. In one embodiment, cefdinir is administered orally. In a further embodiment, the dose of cefdinir is 300 mg or 600 mg per day.

In one embodiment, the second active agent is a carbapenem. The carbapenem in one embodiment, is imipenem, doripenem, biapenem or tebipenem. In a further embodiment, imipenem is used in the one of the methods described herein as the second active agent. Imipenem is a β-lactam antibiotic that has been found to be synergistic with amikacin against certain NTM strains, and as such, can be used in the methods described herein. Imipenem, in one embodiment, is administered intravenously to the patient in need of treatment. In one embodiment, imipenem is administered with cilastatin to prevent its inactivation by the renal enzyme dehydropeptidase 1.

Doripenem, a β-lactam antibiotic in the carbapenem class, in one embodiment, is used as the second active agent in one of the NTM lung disease treatment methods provided herein. In one embodiment, doripenem is administered intravenously to a patient in need of NTM lung disease treatment. In a further embodiment, 500 mg doripenem is administered once daily, twice daily or three times daily during the administration period. When doripenem is administered twice daily or three times daily, administration is spaced by 8 hr. intervals.

Biapenem is another carbapenem that can be used in the methods described herein. In one embodiment, biapenem is administered intravenously to a patient in need of NTM lung disease treatment.

Tebipenem is yet another carbapenem that can be used in the methods described herein. In one embodiment, tebipenem is administered orally to a patient in need of NTM lung disease treatment. In one embodiment, tebipenem is administered as the ester tebipenem pivoxil due to its improved absorption and bioavailability, as compared to the non-ester form.

Ethambutol, in one embodiment, is the second active agent used in the methods provided herein. In a further embodiment, ethambutol is administered orally to the patient.

The calcium channel blocker tetrandrine, in one embodiment, is used in the methods provided herein as the second active agent. In a further embodiment, the tetrandrine is administered orally. In even a further embodiment, tetrandrine is administered once-daily. For example, 60 mg tetrandrine can be administered once-daily in the methods provided herein, during the administration period.

The NTM lung disease treated by a method provided herein, in one embodiment, is caused by one of the following NTM species: M. avium complex, M. kansasii, M abscessus, or M. fortuitum. In a further embodiment, the NTM lung disease is caused by M. avium complex. In another embodiment, the NTM lung disease is caused by an M. abscessus lung infection.

In one embodiment, the NTM lung disease treated by the methods provided herein is newly diagnosed and the treatment regimen set forth in the methods provided herein represents a front-line therapy.

The term “newly diagnosed” as used herein, refers to:
(i) an untreated, initially diagnosed NTM lung disease based on a positive sputum culture for an NTM strain, or
(ii) an untreated NTM lung disease diagnosed based on a newly positive sputum culture for the NTM, subsequent to a previously diagnosed NTM lung infection based on a previous positive sputum culture for the NTM, wherein the previously diagnosed NTM lung disease is treated, and the treatment of the previously diagnosed NTM lung disease is ceased when a negative sputum culture is achieved, and wherein the negative sputum culture for the NTM returns to the newly positive sputum culture for NTM at least 6 months after the cessation of the treatment of the previously diagnosed NTM lung disease.

In one embodiment, the patient subjected to one of the treatment methods provided herein is a patient that was previously non-responsive to a different NTM treatment. In other words, in one embodiment, the patient subjected to one of the treatment methods described herein is refractory to a prior treatment.

In another embodiment, the methods provided herein are implemented for the treatment or prophylaxis of one or more NTM pulmonary infections in a CF patient. In a further embodiment, the liposomal amikacin composition administered to the patient in need of treatment is one of the compositions set forth in Table 1 or Table 2, above.

In one embodiment, the patient is a bronchiectasis patient. In one embodiment, the bronchiectasis is non-cystic fibrosis bronchiectasis (NCFBE). In another embodiment, the bronchiectasis is associated with CF.

A patient subjected to the methods described herein, in one embodiment, has a co-morbid condition. For example, in one embodiment, the patient in need of treatment with one of the methods described herein has diabetes, mitral valve disorder (e.g., mitral valve prolapse), acute bronchitis, pulmonary hypertension, pneumonia, asthma, trachea cancer, bronchus cancer, lung cancer, cystic fibrosis, pulmonary fibrosis, a larynx anomaly, a trachea anomaly, a bronchus anomaly, aspergillosis, HIV or bronchiectasis, in addition to the pulmonary NTM infection. In one embodiment, the patient in need of treatment of an NTM lung disease with one of the methods provided herein has been diagnosed with chronic obstructive pulmonary disease (COPD). In yet another embodiment, the patient in need of treatment of the NTM pulmonary infection is an asthma patient. In a further embodiment, the composition administered to the patient in need of treatment is one of the compositions set forth in Table 1 or Table 2, above.

In one embodiment, a patient in need of treatment with one of the methods described herein is a CF patient, a bronchiectasis patient, a ciliary dyskinesia patient, a chronic smoker, a chronic obstructive pulmonary disorder (COPD) patient, or a patient who has been previously non-responsive to treatment. In a further embodiment, a CF patient is treated for an NTM pulmonary infection with one of the methods provided herein. In yet another embodiment, the patient is a bronchiectasis patient, a COPD patient or an asthma patient.

In one embodiment, treating comprises the patient achieving a negative NTM sputum culture. The negative sputum culture can be achieved during the administration period or subsequent to the administration period. The patient sputum sample(s), in one embodiment, is obtained from the patient at regular time intervals during the administration period and/or immediately after the administration period and is subsequently used to prepare the NTM culture(s).

In another embodiment, treating comprises the patient achieving NTM sputum culture conversion to negative, during the administration period or subsequent to the administration period. Sputum culture conversion to negative as provided herein, refers to three consecutive negative NTM cultures. The samples can be taken during the administration period or a combination of during the administration period and subsequent thereto. The three consecutive cultures can be prepared from patient samples that are obtained at spaced apart intervals, for example, two-week or monthly intervals. The time to conversion, in one embodiment, is about 90 days, or about 100 days or about 110 days. In another embodiment, the time to conversion is from about 90 days to about 200 days, from about 90 days to about 190 days, from about 90 days to about 180 days, from about 90 days to about 160 days, from about 90 days to about 150 days, from about 90 days to about 140 days, from about 90 days to about 130 days, from about 90 days to about 120 days, from about 90 days to about 110 days, from about 90 days to about 110 days, or from about 90 days to about 100 days.

In some embodiments, the therapeutic response resulting from the method provided herein is measured by one or more patient reported outcomes (PROs) generated from PRO instruments, such as Quality of Life Questionnaire—Bronchiectasis (QOL-B), Patient Global Impression of Severity (PGIS) Respiratory, PROMIS Fatigue Short Form 7a (PROMIS F-SF 7a), and Patient Global Impression of Severity (PGIS)-Fatigue.

The QOL-B is a validated, self-administered, reported outcome questionnaire used to assess symptoms, functioning, and health related quality of life in adults with non-CF bronchiectasis. See Quittner A L, Abbott J, Georgiopoulos A M, et al. Quality of Life Questionnaire-Bronchiectasis: final psychometric analyses and determination of minimal important differences scores. Thorax. 2016; 71(1):26-34; incorporated herein by reference in its entirety. It measures outcomes over a recall period of 1 week. The questionnaire contains 37 items on 8 scales (physical, role, vitality, emotional, social, treatment burden, health perception, and respiratory). Each of the 37 items is scored from 1 to 4, and each of the 8 scale scores is standardized on a 0 to 100-point scale, with higher scores representing fewer symptoms or better functioning and quality of life. Scores are calculated for the 7 domains: physical, role, vitality, emotional, social, treatment burden, health perception, and respiratory.

In one embodiment of the method disclosed herein, the treating comprises improving one or more respiratory symptoms, as measured by a QOL-B respiratory domain score, for the patient during or subsequent to the administration period, as compared to the one or more respiratory symptoms of the patient prior to the treatment, or as compared to the one or more respiratory symptoms of a patient administered either the liposomal amikacin composition or the second active agent for the same administration period. In one embodiment, the one or more respiratory symptoms for the patient are improved from about three to about six months after the administration period has ended. In another embodiment, the one or more respiratory symptoms for the patient are improved from about three to about twelve months after the administration period has ended.

In one embodiment, the treating comprises improving the score of one or more of the QOL-B non-respiratory domains for the patient during or subsequent to the administration period, as compared to the score of the one or more of the QOL-B non-respiratory domains of the patient prior to the treatment, or as compared to the score of the one or more of the QOL-B non-respiratory domains of a patient administered either the liposomal amikacin composition or the second active agent for the same administration period. In a further embodiment, the score of the one or more of the QOL-B non-respiratory domains for the patient is improved during the administration period. In another embodiment, the score of the one or more of the QOL-B non-respiratory domains for the patient is improved about one month after the administration period ends. In still a further embodiment, the score of the one or more of the QOL-B non-respiratory domains for the patient is improved at least about one month after the administration period ends. In still a further embodiment, the score of the one or more of the QOL-B non-respiratory domains for the patient is improved from about one to about three months after the administration period ends. In still a further embodiment, the score of the one or more of the QOL-B non-respiratory domains for the patient is improved about three months after the administration period ends. In still a further embodiment, the score of the one or more of the QOL-B non-respiratory domains for the patient is improved at least about three months after the administration period ends. In still a further embodiment, the score of the one or more of the QOL-B non-respiratory domains for the patient is improved from about three to about six months after the administration period ends. In still a further embodiment, the score of the one or more of the QOL-B non-respiratory domains for the patient is improved from about three to about twelve months after the administration period ends.

The PGIS Respiratory score is a simple categorical rating of symptom severity. The scale is 0=not at all to 5=extremely severe. In one embodiment of the method disclosed herein, the treating comprises improving one or more respiratory symptoms, as measured by a PGIS Respiratory score, for the patient during or subsequent to the administration period, as compared to the one or more respiratory symptoms of the patient prior to the treatment, or as compared to the score of the PGIS Respiratory score of a patient administered either the liposomal amikacin composition or the second active agent for the same administration period. In a further embodiment, the one or more respiratory symptoms for the patient are improved during the administration period. In still a further embodiment, the one or more respiratory symptoms for the patient are improved subsequent to the administration period. In still a further embodiment, the one or more respiratory symptoms for the patient are improved about one month after the administration period ends. In still a further embodiment, the one or more respiratory symptoms for the patient are improved at least about one month after the administration period ends. In another embodiment, the one or more respiratory symptoms for the patient are improved from about one to about three months after the administration period ends. In another embodiment, the one or more respiratory symptoms for the patient are improved about three months after the administration period ends. In yet another embodiment, the one or more respiratory symptoms for the patient are improved at least about three months after the administration period ends. In another embodiment, the one or more respiratory symptoms for the patient are improved from about three to about six months after the administration period ends. In another embodiment, the one or more respiratory symptoms for the patient are improved from about three to about twelve months after the administration period ends.

The PROMIS F-SF 7a is a self-administered questionnaire assessing a range of self-reported symptoms over the past 7 days, from mild subjective feelings of tiredness to an overwhelming, debilitating, and sustained sense of exhaustion that likely decreases one's ability to execute daily activities and function normally in family or social roles. See Ameringer S, Elswick R K, Jr., Menzies V, et al. Psychometric Evaluation of the Patient-Reported Outcomes Measurement Information System Fatigue-Short Form Across Diverse Populations. Nurs Res. 2016; 65(4):279-289, incorporated herein by reference in its entirety. Fatigue is divided into the experience of fatigue (frequency, duration, and intensity) and the impact of fatigue on physical, mental, and social activities over 7 items. Response options are on a 5-point Likert scale, ranging from 1=never to 5=always. The PROMIS F-SF 7a is universal rather than disease-specific.

In one embodiment of the method disclosed herein, the treating comprises improving one or more fatigue symptoms, as measured by a PROMIS F-SF 7a score, for the patient during or subsequent to the administration period, as compared to the one or more fatigue symptoms of the patient prior to the treatment, or as compared to the one or more fatigue symptoms of a patient administered either the liposomal amikacin composition or the second active agent for the same administration period. In one embodiment, the one or more fatigue symptoms for the patient are improved during the administration period. In one embodiment, the one or more fatigue symptoms for the patient are improved at the end of the administration period. In another embodiment, the one or more fatigue symptoms for the patient are improved about one month after the administration period ends. In still a further embodiment, the one or more fatigue symptoms for the patient are improved at least about one month after the administration period ends. In still a further embodiment, the one or more fatigue symptoms for the patient are improved from about one to about three months after the administration period ends. In still a further embodiment, the one or more fatigue symptoms for the patient are improved about three months after the administration period ends. In still a further embodiment, the one or more fatigue symptoms for the patient are improved at least about three months after the administration period ends. In still a further embodiment, the one or more fatigue symptoms for the patient are improved from about three to about six months after the administration period ends. In still a further embodiment, the one or more fatigue symptoms for the patient are improved from about three to about twelve months after the administration period ends.

The PGIS Fatigue score is a simple categorical rating of symptom severity. The scale is 0=not at all to 5=extremely severe.

In one embodiment of the method disclosed herein, the treating comprises improving one or more fatigue symptoms, as measured by a PGIS Fatigue score, for the patient during or subsequent to the administration period, as compared to the one or more fatigue symptoms of the patient prior to the treatment, or as compared to the one or more fatigue symptoms of a patient administered either the liposomal amikacin composition or the second active agent for the same administration period. In a further embodiment, the one or more fatigue symptoms for the patient are improved during the administration period. In still a further embodiment, the one or more fatigue symptoms for the patient are improved at the end of the administration period. In still a further embodiment, the one or more fatigue symptoms for the patient are improved about one month after the administration period ends. In still a further embodiment, the one or more fatigue symptoms for the patient are improved at least about one month after the administration period ends. In still a further embodiment, the one or more fatigue symptoms for the patient are improved from about one to about three months after the administration period ends. In still a further embodiment, the one or more fatigue symptoms for the patient are improved about three months after the administration period ends. In still a further embodiment, the one or more fatigue symptoms for the patient are improved at least about three months after the administration period ends. In still a further embodiment, the one or more fatigue symptoms for the patient are improved from about three to about six months after the administration period ends. In still a further embodiment, the one or more fatigue symptoms for the patient are improved from about three to about twelve months after the administration period ends.

In some embodiments, the patient experiences an improvement in lung function for at least 15 days after the administration period ends, as compared to the lung function of the patient prior to treatment. For example, the patient may experience an increase in FEV₁, an increase in blood oxygen saturation, or both. In some embodiments, the patient has an FEV₁ (after the administration period or treatment cycle) that is increased by at least 5% over the FEV₁ prior to the administration period. In other embodiments, FEV₁ is increased by 5 to 50% over the FEV₁ prior to the administration period. In other embodiments, FEV₁ is increased by 25 to 500 mL over FEV₁ prior to the administration period. In some embodiments, blood oxygen saturation is increased by at least 1% over oxygen saturation prior to the administration period.

In one embodiment, the 6-minute walk test (6MWT) is used to assess the effectiveness of the treatment methods provided herein. The 6MWT is used for the objective evaluation of functional exercise capacity and is a practical, simple test that measures the distance that a patient can walk in a period of 6 minutes (see American Thoracic Society. (2002). Am J Respir Crit Care Med. 166, pp. 111-117, incorporated by reference herein in its entirety for all purposes).

In one embodiment, a patient subjected to one of the NTM methods described herein exhibits an increased number of meters walked in the 6MWT, as compared to prior to undergoing the treatment method. The increased number of meters walked in the 6MWT, in one embodiment, is about 5 meters, about 10 meters, about 15 meters, about 20 meters, about 25 meters, about 30 meters, about 35 meters, about 40 meters, about 45 meters, or about 50 meters. In another embodiment, the increased number of meters walked in the 6MWT is at least about 5 meters, at least about 10 meters, at least about 15 meters, at least about 20 meters, at least about 25 meters, at least about 30 meters, at least about 35 meters, at least about 40 meters, at least about 45 meters, or at least about 50 meters. In yet another embodiment, the increased number of meters walked in the 6MWT is from about 5 meters to about 50 meters, or from about 5 meters to about 40 meters, or from about 5 meters to about 30 meters or from about 5 meters to about 25 meters.

In another embodiment, a patient subjected to one of the NTM methods described herein exhibits a greater number of meters walked in the 6MWT, as compared to a patient undergoing treatment with either the liposomal amikacin composition or the second active agent for the same administration period. The greater number of meters walked in the 6MWT, in one embodiment, is about 5 meters, about 10 meters, about 15 meters, about 20 meters, about 25 meters, about 30 meters, about 35 meters, about 40 meters, about 45 meters, about 50 meters, about 60 meters, about 70 meters or about 80 meters. In another embodiment, the greater number of meters walked in the 6MWT is at least about 5 meters, at least about 10 meters, at least about 15 meters, at least about 20 meters, at least about 25 meters, at least about 30 meters, at least about 35 meters, at least about 40 meters, at least about 45 meters, or at least about 50 meters. In yet another embodiment, the greater number of meters walked in the 6MWT is from about 5 meters to about 80 meters, or from about 5 meters to about 70 meters, or from about 5 meters to about 60 meters or from about 5 meters to about 50 meters.

### EXAMPLE

The present invention is further illustrated by reference to the following Example. However, it should be noted that this Example, like the embodiments described above, is illustrative and are not to be construed as restricting the scope of the invention in any way.

### Example 1: Synergistic Combinations of Antiinfectives Against Non-Tuberculous Mycobacterium

To assay for synergistic combinations of active agents against nontuberculous mycobacterium (NTM), a checkerboard minimum inhibitory concentration (MIC) assay is carried out to determine the impact on potency of certain combination of active agents in comparison to individual MICs. The comparison is represented as the Fractional Inhibitory Concentration Index (FICI), which takes into account the combination of active agents that produces greatest change from the MIC of the individual active agent. In the example provided herein, MIC is assessed for M abscessus ATCC 19977 Type strain. However, these methods are applicable to other NTM species described herein.

### Assay Setup and Reagents

The following protocol is carried out for each pair of active agents (referred to as Active Agent-X and Active Agent-Y in this section).

Fresh cultures of slow-growing NTM strain (exemplified here with M abscessus ATCC 19977) were prepared on 7H10+10% Oleic Albumin Dextrose Catalase (OADC) agar or in 7H9+10% OADC broth.

About 5×10⁸ CFU/mL inoculum of bacteria was prepared in Hanks' Balanced Salt Solution (HBSS) using standard protocols.

The inoculum suspension was diluted 1:10 into HBSS and then 1:100 (total 1:1,000 dilution to achieve 5×10⁵ colony forming units (CFU)/mL) into the respective volume of broth needed, which was 15 mL per checkerboard plate.

Checkerboard plate wells were filled with 100 μL of the diluted inoculum broth suspension (100 μL per well). All plate wells in rows B-H were filled, and row A was initially left empty.

Active Agent-X was diluted in pre-inoculated broth such that the highest dose to be tested of Active Agent-X was present in 100 μL of the broth. The dilution was carried out in a 5 mL culture tube.

100 μL aliquots of the Active Agent-X dilution were added to each well in row A of the checkerboard plate using a multipipette.

100 μL of the Active Agent-X dilution (i.e., the highest dose to be tested) was added to row B. Active Agent-X was mixed into row B 10× via pipette.

The row B contents were then serially diluted 1:1 via 10× mixing down all rows through row G. 100 μL of row G was then discarded, yielding 100 μL in all wells of the plate.

Wells in row H were left empty.

Active Agent-Y was diluted in pre-inoculated broth such that 2× the highest dose to be tested of Active Agent-Y was present in 100 μL of the broth. The dilution was carried out in a 5 mL culture tube.

100 μL of the Active Agent-Y stock was added to each well of column 1 (8 wells total).

Using a multichannel pipette, the contents of each well of column 1 were mixed 10×, and 100 μL of the column 1 contents were transferred into column 2.

The contents of column 2 were serially diluted 1:1 via 10× mixing across each column through column 11. 100 μL of column 11 was discarded after mixing, yielding 100 μL in all wells of the plate. No material was added to column 12 at this step.

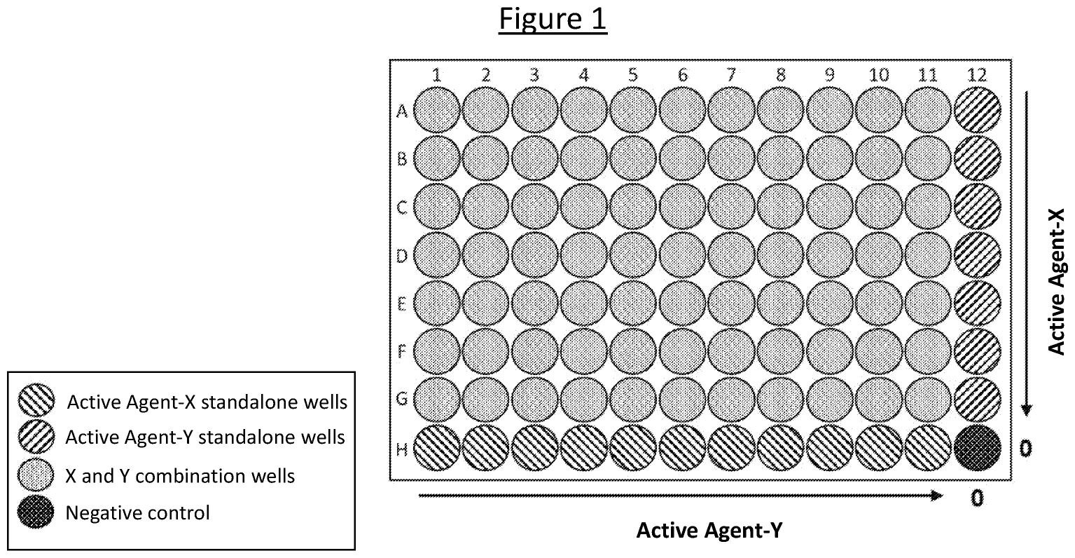

At this stage, the checkerboard plate was fully filled with (i) the Active Agent-X stand-alone MIC being column 12, (ii) the Active Agent-Y stand-alone MIC being row H, (iii) the negative (no drug) control being well H12, and the mixed drug matrix of concentrations in all the other wells of the plate (see schematic at FIG. 1).

The checkerboard plates were placed into ziptop bags and incubated at 37° C. for 7 days unless otherwise noted.

Absorbance values for each well were taken with plate reader (BioTek Synergy H1) at 595 nm after the 7-day incubation period. MIC values were determined based on absorbance values. FICI values were then calculated as follows.

### FICI Measurements

FICI is determined by the following equation.

$$\frac{A}{MICA} + \frac{B}{MICB} = FICA + FICB = F I C I$$

where A and B are the MIC of Active Agent-X and Active Agent-Y in combination (in a single well), and MICA and MICB are the MIC of each antiinfective individually.

TABLE 4

| FIC value | Interpretation |
|---|---|
| <0.5 | Synergy |
| >4 | Antagonism |
| 0.5-4 | Additive or indifference |

Combinations are determined to be synergistic based on the values provided above in Table 4.

Table 5 provides the results for all active agent combinations tested.

TABLE 5

| | Amikacin | | | | | |
|---|---|---|---|---|---|---|
| | (Active Agent-X) | | Active Agent-Y | | | |
| | MIC (μg/ML) | | MIC (μg/ML) | | | |
| Antiinfective-Y | Alone | Combination | Alone | Combination | FICI | Definition |
| Biapenem | 2 | 0.125 | 8 | 0.0625 | 0.07 | Synergy |
| Bedaquiline | 2 | 0.0625 | 2 | 0.125 | 0.09 | Synergy |
| Cefdinir | 2 | 0.125 | 8 | 0.5 | 0.13 | Synergy |
| Levofloxacin | 2 | 0.25 | 4 | 0.0625 | 0.14 | Synergy |
| Clofazimine | 2 | 0.25 | 2 | 0.0625 | 0.16 | Synergy |
| Tetrandrine | 1 | 0.125 | 64 | 8 | 0.25 | Synergy |
| Tebipenem | 2 | 0.5 | 8 | 0.25 | 0.28 | Synergy |
| Doripenem | 2 | 0.5 | 4 | 0.25 | 0.31 | Synergy |
| Rifabutin | 2 | 0.5 | 1 | 0.0625 | 0.31 | Synergy |
| Rifampicin | 2 | 0.5 | 8 | 0.5 | 0.31 | Synergy |
| RV40¹ | 2 | 0.5 | 16 | 1 | 0.31 | Synergy |
| Moxifloxacin | 2 | 0.5 | 1 | 0.125 | 0.38 | Synergy |
| Imipenem | 2 | 0.5 | 2 | 0.25 | 0.38 | Synergy |
| Ethambutol | 2 | 0.25 | 16 | 4 | 0.38 | Synergy |
| Vancomycin | 2 | 0.5 | 4 | 1 | 0.50 | Additive |
| Tigecycline | 2 | 1 | 2 | 0.5 | 0.75 | Additive |
| Clarithromycin | 2 | 4 | 0.5 | 0.25 | 2.50 | Additive |
| Linezolid | 1 | 2 | 2 | 2 | 3.00 | Additive |
| Tedizolid | 1 | 2 | 0.5 | 0.5 | 3.00 | Additive |

¹See WO 2018/217800, incorporated by reference herein in its entirety, for the structure of RV40 and methods of making the same.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described. Accordingly, the foregoing descriptions and drawings are by way of example only and the disclosure is described in detail by the claims that follow.

1. A method for treating a nontuberculous mycobacterium (NTM) lung disease in a patient in need of treatment, comprising:
administering to the patient for an administration period (i) a liposomal amikacin composition and (ii) a second active agent that is synergistic with amikacin against the NTM,
wherein the liposomal amikacin composition comprises amikacin, or a pharmaceutically acceptable salt thereof, encapsulated in a plurality of liposomes, wherein the lipid component of the plurality of liposomes consists of one or more electrically neutral lipids,
wherein the liposomal amikacin composition is administered to the patient once-daily in a single dosing session, and administering comprises aerosolizing the liposomal amikacin composition to provide an aerosolized composition comprising a mixture of free amikacin and liposomal complexed amikacin, and administering the aerosolized pharmaceutical composition via a nebulizer to the lungs of the patient via inhalation, and
wherein the second active agent is administered orally, parenterally or via inhalation.

2. The method of claim 1, wherein the amikacin or pharmaceutically acceptable salt thereof is amikacin.

3. The method of claim 1, wherein the amikacin or pharmaceutically acceptable salt thereof is amikacin sulfate.

4. The method of any one of claims 1-3, wherein the one or more electrically neutral lipids comprises an (i) electrically neutral phospholipid or (ii) an electrically neutral phospholipid, and a sterol.

5. The method of any one of claims 1-3, wherein the one or more electrically neutral lipids consists of a phosphatidylcholine and a sterol.

6. The method of any one of claims 1-3, wherein the one or more electrically neutral lipids comprises dipalmitoylphosphatidylcholine (DPPC) and a sterol.

7. The method of any one of claims 1-3, wherein the one or more electrically neutral lipids comprises DPPC and cholesterol.

8. The method of any one of claims 1-7, wherein the plurality of liposomes comprises unilamellar vesicles, multilamellar vesicles, or a mixture thereof.

9. The method of any one of claims 1-8, wherein the liposomal amikacin composition is a liposomal suspension having a volume of from about 8 mL to about 10 mL.

10. The method of any one of claims 1-9, wherein the liposomal amikacin composition comprises from about 500 mg to about 650 mg amikacin, or pharmaceutically acceptable salt thereof, or from about 550 mg to about 625 mg amikacin, or pharmaceutically acceptable salt thereof, or from about 550 mg to about 600 mg amikacin, or pharmaceutically acceptable salt thereof.

11. The method of any one of claims 1-10, wherein the liposomal amikacin composition comprises about 65 to about 80 mg/mL amikacin, or pharmaceutically acceptable salt thereof, about 25 to about 35 mg/mL DPPC; and about 10 to about 20 mg/mL cholesterol.

12. The method of claim 11, wherein the liposomal amikacin composition comprises about 65 to about 75 mg/mL amikacin sulfate; about 30 to about 35 mg/mL DPPC; and about 15 to about 19 mg/mL cholesterol.

13. The method of any one of claims 9-12, wherein the liposomal amikacin composition has a volume of from about 8 mL to about 9 mL.

14. The method of any one of claims 1-13, wherein during the single dosing session, the aerosolized composition is administered via the nebulizer in less than about 15 minutes, less than about 14 minutes, less than about 13 minutes, less than about 12 minutes, or less than about 11 minutes.

15. The method of any one of claims 1-13, wherein during the single dosing session, the aerosolized composition is administered via the nebulizer in about 10 minutes to about 14 minutes, about 10 minutes to about 13 minutes, about 10 minutes to about 12 minutes, about 10 minutes to about 11 minutes, about 11 minutes to about 15 minutes, about 12 minutes to about 15 minutes, about 13 minutes to about 15 minutes or about 14 minutes to about 15 minutes.

16. The method of any one of claims 1-15, wherein the second active agent is administered orally.

17. The method of any one of claims 1-15, wherein the second active agent is administered parenterally.

18. The method of claim 17, wherein the second active agent is administered intravenously.

19. The method of any one of claims 1-15, wherein the second active agent is administered via inhalation.

20. The method of claim 19, wherein the second active agent is present in the liposomal amikacin composition.

21. The method of claim 19, wherein the second active agent is administered in a separate composition from the liposomal amikacin composition.

22. The method of claim 21, wherein the second active agent is administered via a nebulizer.

23. The method of claim 21, wherein the second active agent is administered via a metered dose inhaler (MDI).

24. The method of claim 21, wherein the second active agent is administered via a dry powder inhaler (DPI).

25. The method of any one of claims 16-24, wherein the second active agent is an antiinfective.

26. The method of any one of claims 16-24, wherein the second active agent is a carbapenem.

27. The method of claim 26, wherein the carbapenem is imipenem, doripenem, biapenem or tebipenem.

28. The method of any one of claims 16-24, wherein the second active agent is rifabutin, rifampin, RV40, clofazimine, bedaquiline, or cefdinir.

29. The method of any one of claims 16-24, wherein the second active agent is imipenem, rifabutin, rifampin, RV40, clofazimine, bedaquiline, cefdinir, doripenem, biapenem, or tebipenem.

30. The method of any one of claims 16-24, wherein the second active agent is imipenem.

31. The method of any one of claims 16-24, wherein the second active agent is rifabutin.

32. The method of any one of claims 16-24, wherein the second active agent is rifampin.

33. The method of any one of claims 16-24, wherein the second active agent is RV40.

34. The method of any one of claims 16-24, wherein the second active agent is clofazimine.

35. The method of any one of claims 16-24, wherein the second active agent is bedaquiline.

36. The method of any one of claims 16-24, wherein the second active agent is cefdinir.

37. The method of any one of claims 16-24, wherein the second active agent is doripenem.

38. The method of any one of claims 16-24, wherein the second active agent is biapenem.

39. The method of any one of claims 16-24, wherein the second active agent is tebipenem.

40. The method of any one of claims 1-39, wherein the patient has cystic fibrosis.

41. The method of any one of claims 1-39, wherein the patient has bronchiectasis.

42. The method of claim 41, wherein the patient has non-cystic fibrosis bronchiectasis.

43. The method of any one of claims 1-42, wherein the patient is a smoker or has a previous history of smoking.

44. The method of any one of claims 1-43, wherein the patient has chronic obstructive pulmonary disorder (COPD).

45. The method of any one of claims 1-44, wherein the patient has asthma.

46. The method of any one of claims 1-45, wherein the patient is a ciliary dyskinesia patient.

47. The method of any one of claims 1-46, wherein the NTM lung disease is caused by M. avium, M. abscessus, M. chelonae, M. bolletii, M. kansasii, M. ulcerans, M. avium complex (MAC) (M. avium and M. intracellulare), M. conspicuum, M. peregrinum, M. immunogenum, M. xenopi, M. malmoense, M. marinum, M. mucogenicum, M. nonchromogenicum, M. scrofulaceum, M. simiae, M. smegmatis, M. szulgai, M. terrae, M. terrae complex, M. haemophilum, M. genavense, M. asiaticum, M. shimoidei, M. gordonae, M. triplex, M. lentiflavum, M. celatum, M. fortuitum, M. fortuitum complex (M. fortuitum and M. chelonae), or a combination thereof.

48. The method of any one of claims 1-46, wherein the NTM lung disease is caused by M. avium.

49. The method of claim 48, wherein the M. avium is M. avium subsp. hominissuis.

50. The method of any one of claims 1-46, wherein the NTM lung disease is caused by Mycobacterium abscessus.

51. The method of any one of claims 1-46, wherein the NTM lung disease is caused by Mycobacterium avium complex (M. avium and M. intracellulare).

52. The method of any one of claims 1-51, wherein the patient in need of treatment was previously unresponsive to NTM therapy.

53. The method of any one of claims 1-51, wherein the patient in need of treatment is a newly diagnosed NTM lung disease patient.

54. The method of any one of claims 1-53, wherein during the administration period, the liposomal amikacin composition and second active agent are administered at the same dosing intervals.

55. The method of any one of claims 1-53, wherein during the administration period, the liposomal amikacin composition and second active agent are administered at different dosing intervals.

56. The method of any one of claims 1-53, wherein during the administration period, the liposomal amikacin composition and second active agent are administered for different durations.

57. The method of any one of claims 1-53, wherein during the administration period, the liposomal amikacin composition and second active agent are administered for the same duration.

58. The method of any one of claims 1-57, wherein the administration period is at least 3 months, at least 4 months, at least 5 months, at least 6 months, at least 9 months, at least 12 months, at least 15 months, at least 18 months or at least 24 months.

59. The method of any one of claims 1-57, wherein the administration period is from about 6 months to about 24 months.

60. The method of any one of claims 1-57, wherein the administration period is from about 6 months to about 18 months

61. The method of any one of claims 1-57, wherein the administration period is from about 6 months to about 12 months.

62. The method of any one of claims 1-57, wherein the administration period is from about 30 days to about 400 days.

63. The method of claim 62, wherein the administration period is from about 45 days to about 300 days, or from about 45 days to about 270 days, or from about 80 days to about 200 days.

64. The method of claim 62, wherein the administration period is from about 80 days to about 400 days, or from about 90 days to about 400 days, or from about 100 days to about 400 days.

65. The method of any one of claims 1-57, wherein the administration period is from about 100 days to about 500 days.

66. The method of any one of claims 1-65, wherein during the administration period, or subsequent to the administration period, the patient exhibits a negative NTM sputum culture.

67. The method of any one of claims 1-65, wherein during the administration period, or subsequent to the administration period, the patient exhibits an NTM sputum culture conversion to negative.

68. The method of claim 67, wherein the time to NTM sputum culture conversion to negative is about 60 days, about 70 days, about 80 days, about 90 days, about 100 days, about 110 days, about 120 days, about 150 days, about 200 days, about 250 days, about 300 days, about 350 days or about 400 days.

69. The method of claim 67, wherein the time to NTM sputum culture conversion to negative is from about 60 days to about 400 days, from about 60 days to about 350 days, from about 60 days to about 300 days, from about 60 days to about 250 days, from about 60 days to about 200 days, from about 60 days to about 150 days, from about 60 days to about 140 days, from about 60 days to about 130 days, from about 60 days to about 120 days, from about 60 days to about 110 days, or from about 60 days to about 100 days.

70. The method of claim 67, wherein the time to NTM sputum culture conversion to negative is from about 90 days to about 400 days, from about 90 days to about 350 days, from about 90 days to about 300 days, from about 90 days to about 250 days, from about 90 days to about 200 days, from about 90 days to about 150 days, from about 90 days to about 140 days, from about 90 days to about 130 days, from about 90 days to about 120 days, from about 90 days to about 110 days, or from about 90 days to about 100 days.

71. The method of any one of claims 1-70, wherein during the administration period, or subsequent to the administration period, the patient shows improvement in one or more respiratory symptoms, as measured by a QOL-B respiratory domain score, as compared to the one or more respiratory symptoms of the patient prior to the treatment.

72. The method of any one of claims 1-71, wherein during the administration period or subsequent to the administration period, the patient exhibits an increased number of meters walked in the 6 minute walk test (6MWT), as compared to the number of meters walked by the patient prior to the treatment.

73. The method of claim 72, wherein the increased number of meters walked in the 6MWT is at least about 5 meters.

74. The method of claim 72, wherein the increased number of meters walked in the 6MWT is at least about 10 meters.

75. The method of claim 72, wherein the increased number of meters walked in the 6MWT is at least about 20 meters.

76. The method of claim 72, wherein the increased number of meters walked in the 6MWT is at least about 30 meters.

77. The method of claim 72, wherein the increased number of meters walked in the 6MWT is at least about 40 meters.

78. The method of claim 72, wherein the increased number of meters walked in the 6MWT is at least about 50 meters.

79. The method of claim 72, wherein the increased number of meters walked in the 6MWT is from about 5 meters to about 50 meters.

80. The method of claim 72, wherein the increased number of meters walked in the 6MWT is from about 15 meters to about 50 meters.
